(19) United States
(12) Patent Application Publication
(10) Pub. No.: US 2012/0191133 A1
(43) Pub. Date: Jul. 26, 2012

(54) WELDABLE FLEXIBLE LONGITUDINAL FIXATION COMPONENTS AND INSTRUMENTS ASSOCIATED THEREWITH

(75) Inventor: Bret A. Ferree, Cincinnati, OH (US)

(73) Assignee: Anova Corporation

(21) Appl. No.: 13/314,570

(22) Filed: Dec. 8, 2011

Related U.S. Application Data
(60) Provisional application No. 61/420,837, filed on Dec. 8, 2010.
(60) Provisional application No. 61/537,839, filed on Sep. 22, 2011.

(51) Int. Cl. A61B 17/04; B32B 37/14

(52) U.S. Cl. 606/228; 156/539

(57) ABSTRACT

Methods and devices facilitate welding sutures during laparoscopic and robotic surgical procedures. A first end of a weldable suture is reversibly fastened to the distal end of a novel welding instrument then inserted into a patient, preferably through a laparoscopic portal or trocar. A needle on the second or distal end of the suture is passed through tissue then the distal end of the suture is reversibly fastened to the distal end of the novel welding instrument. An alignment feature in the distal end of the instrument aligns portions of the suture for placement in the jaws of a welding tool. The surgeon may tighten the suture by pulling on the ends of the suture, which reduces the diameter of the suture loop and pulls the tissues together. The instrument maintains tension on the suture and immobilizes the suture during suture welding. Sutures with low melting temperatures may be melted into and around portions of rapid absorption multifilament suture.

### REFERENCE TO RELATED APPLICATIONS

This application claims priority to U.S. Provisional Patent Application Ser. Nos. 61/420,837, filed Dec. 8, 2010 and 61/537,839, filed Sep. 22, 2011; the entire content of both applications being incorporated herein by reference.

### FIELD OF THE INVENTION

This invention relates generally to welding sutures and, in particular, to apparatus and methods for welding sutures made of components that have different melting temperatures and generally have different absorption times. The invention also facilitates laparoscopic and da Vinci-aided surgeries.

### BACKGROUND OF THE INVENTION

Sutures optimized for thermal welding have multifilament cores made of high melting temperature fibers and sheaths made of fibers that melt at temperatures at least 40 degrees Centigrade below the melting temperatures of the core fibers. We discovered prior art absorbable sutures can not be welded with thermal welders or such welds fail with small tensile loads.

Fibers made of polyglycolic acid (Dexon II, Covidien, Dubin Ireland), polyglactin (Vicryl, Ethicon, Somerville, NA glycolide/lacti de copolymer (Lactomer, Polysorb, Covidien, Dublin, Ireland), or Polyglytone 621 (Caprosyn, Covidien, Dublin Ireland) absorb within 90 days. Generally such materials have melting temperatures between about 150° C. and 230° C. Fibers made of copolymer materials with 90 to 120 day absorption times, such as Biosyn (Covidien, Dublin Ireland) and Monocryl (Ethicon, Somerville, N.J.) melt at about 210 to 220° C. Fibers made of materials with low melting temperatures, generally between 60° C. and 106° C., such as TephaFLEX (Tepha Medical Devices, Lexington Mass., 60° C.) and Monoplus (B. Braun, Bethlehem, Pa., 106° C.) have 180 day and 300 day absorption times, respectively.

Based upon melting temperatures, Tepha and Monoplus materials are ideal materials for sheath components of bi-composite weldable sutures. However, absorption times of such materials are too long for many surgical procedures, such as laparoscopic assisted hysterectomy. Surgeons require rapid absorption sutures, such as Dexon II and Vicryl and Polysorb for such procedures.

There are now a wide variety of minimally invasive surgical procedures and robotic systems that facilitate such procedures. The da Vinci System (Intuitive Surgical, Sunnyvale, Calif.), for example, is a robotic platform that offers a minimally invasive option for major surgery. With da Vinci system, small incisions are used to introduce miniaturized, wristed instruments and a high-definition 3D camera that a surgeon uses to view a magnified, high-resolution 3D image of the surgical site. Robotic and computer hardware and software translate the surgeon's hand movements into precise micro-movements of the da Vinci instruments. Even with systems of this type, many surgeons struggle trying to tie knots laparoscopically. Many surgeons believe that weldable sutures and instruments that facilitate suture welding through laparoscopic trocars would decrease operation times by as much as 50 percent.

### SUMMARY OF THE INVENTION

This invention teaches methods and devices to facilitate welding sutures during laparoscopic procedures and procedures aided by robotic systems that facilitate minimally invasive procedures, including the da Vinci Surgical System.

In accordance with one embodiment, a first or proximal end of a weldable suture is reversibly fastened to the distal end of a novel welding instrument then inserted into a patient, preferably through a laparoscopic portal or trocar. A needle on the second or distal end of the suture is passed through tissue then the distal end of the suture is reversibly fastened to the distal end of the novel welding instrument. An alignment feature in the distal end of the instrument aligns portions of the suture for placement in the jaws of a welding tool.

In preferred embodiments, apparatus is provided enabling surgeons to tighten the suture by pulling on the ends of the suture, which reduces the diameter of the suture loop and pulls the tissues together. The instrument maintains tension on the suture and immobilizes the suture during suture welding. The instrument can be removed and reloaded for additional suture welds.

Welding devices according to the invention may be configured for attachment to the arms of surgical robots, including the da Vinci Surgical System. The invention also teaches methods and apparatus for welding absorbable sutures. For example, sutures with low melting temperatures are preferably melted into and around portions of rapid absorption multifilament suture. The invention limits the melted suture material, which generally has long absorption times, to the exterior of the closed tissue.

### BRIEF DESCRIPTION OF THE DRAWINGS

FIG. 1A is an anterior view of the preferred embodiment of the invention;

FIG. 1B is a lateral view of the embodiment of the invention drawn in FIG. 1A;

FIG. 1C is an exploded anterior view of the embodiment of the invention drawn in FIG. 1A;

FIG. 1D is an anterior view of the embodiment of the invention drawn in FIG. 1A, a flexible longitudinal fixation component, and a cross section of tissue;

FIG. 1E is an anterior view of the embodiment of the invention drawn in FIG. 1D, a flexible longitudinal fixation component, and tissue;

FIG. 1F is an anterior view of the embodiment of the invention drawn in FIG. 1E, a flexible longitudinal fixation component, and a cross section of tissue;

FIG. 1G is a lateral view of the embodiment of the invention drawn in FIG. 1F, a flexible longitudinal fixation component, and tissue;

FIG. 2A is an anterior view of an alternative embodiment of the invention drawn in FIG. 1A and a flexible longitudinal fixation component;

FIG. 2B is an anterior view of the embodiment of the invention drawn in FIG. 2A and tissue;

FIG. 3A is an anterior view of the distal end of an alternative embodiment of the invention drawn in FIG. 2A and two flexible longitudinal fixation components;

FIG. 3B is an anterior view of the distal end of the embodiment of the invention drawn in FIG. 3A, two flexible longitudinal fixation components, and tissue;

FIG. 3C is an anterior view of a longitudinal cross section of the distal end of the embodiment of the invention drawn in FIG. 3B, two flexible longitudinal fixation components, and tissue;

FIG. 3D is an anterior view of a cross section of the flexible longitudinal fixation components and tissue drawn in FIG. 3C;

FIG. 3E is a superior view of a component of the invention drawn in FIG. 3A;

FIG. 3F is an inferior view of the component drawn in FIG. 3E;

FIG. 3G is an anterior view of a cross section of the component drawn in FIG. 3F;

FIG. 3H is an anterior view of a longitudinal cross section of the embodiment of the invention drawn in FIG. 3A and a tool used to load the device;

FIG. 4A is a lateral view of an embodiment of the invention similar to the inventions drawn in FIGS. 1A and 1D of my co-pending application Ser. No. 61/420,837 entitled “Weldable Flexible Longitudinal Fixation Components”;

FIG. 4B is an anterior view of the distal end of an alternative embodiment of the invention drawn in FIG. 3A and the embodiment of the invention drawn in FIG. 4A;

FIG. 5A is an anterior view of the distal end of an alternative embodiment of the invention drawn in FIG. 3A;

FIG. 5B is a posterior view of the distal end of the embodiment of the invention drawn in FIG. 5A;

FIG. 5C is a inferior view of a transverse cross section of the embodiment of the invention drawn in FIG. 5B;

FIG. 5D is a inferior view of a transverse cross section of an alternative embodiment of the invention drawn in FIG. 5C;

FIG. 6A is an oblique anterior view of an alternative embodiment of the invention flexible longitudinal fixation component drawn in FIG. 1D;

FIG. 6B is an anterior view of the embodiment of the invention drawn in FIG. 6A and tissue;

FIG. 6C is an anterior view of a cross section of the embodiment of the invention drawn in FIG. 6B and tissue;

FIG. 6D is an anterior view of an alternative of the embodiment of the invention drawn in FIG. 6C;

FIG. 7A is a lateral view of an alternative embodiment of the invention drawn in FIG. 6A;

FIG. 7B is a lateral view of the embodiment of the invention drawn in FIG. 7A and tissue;

FIG. 7C is a lateral view of the embodiment of the invention drawn in FIG. 7B and tissue;

FIG. 7D is a lateral view of the embodiment of the invention drawn in FIG. 7C and tissue;

FIG. 8A is a lateral view of an alternative embodiment of the invention drawn in FIG. 7A;

FIG. 8B is a superior view of flexible longitudinal fixation component threaded through tissue;

FIG. 8C is a superior view of the flexible longitudinal fixation component and tissue drawn in Figure 8B;

FIG. 8D is a superior view of a flexible longitudinal fixation component, tissue and the embodiment of the invention drawn in FIG. 8A;

FIG. 8E is a superior view of the flexible longitudinal fixation component, tissue and embodiment of the invention drawn in FIG. 8D; and

FIG. 8F is a superior view of the flexible longitudinal fixation component, tissue and embodiment of the invention drawn in FIG. 8E:

### DETAILED DESCRIPTION OF THE INVENTION

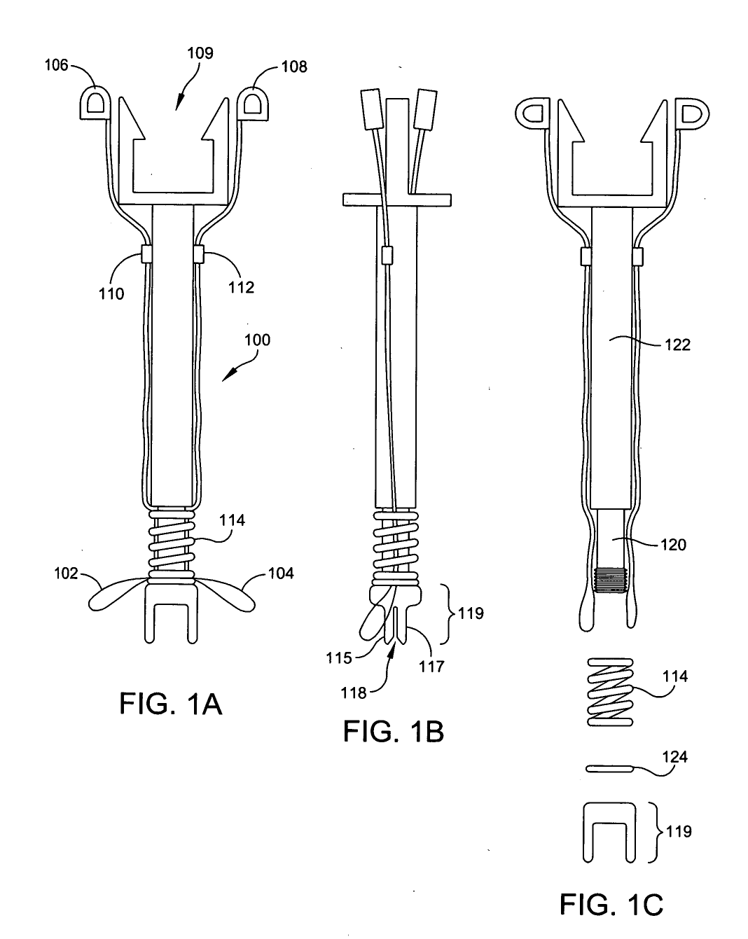

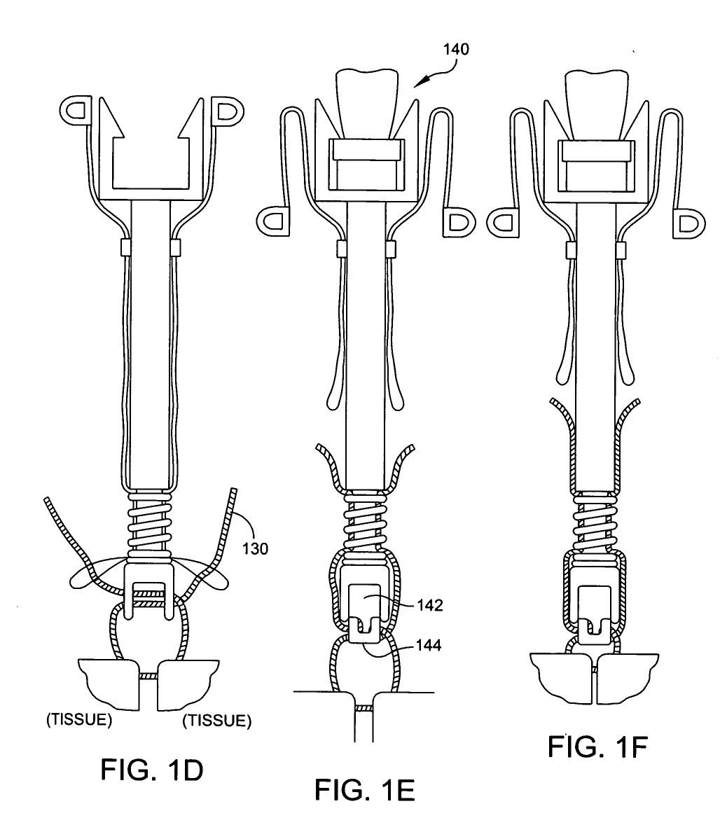

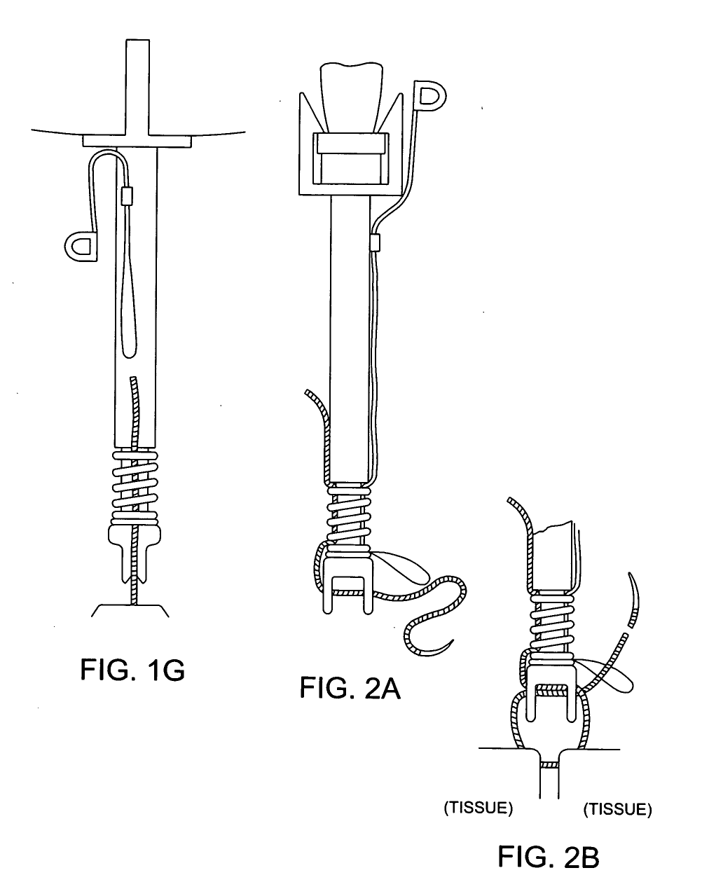

FIG. 1A is an anterior view of the preferred embodiment of the invention. Two wire or cable loops 102, 104 are seen along the lateral sides of device 100. The cable loops are preferably made of multiple filaments of stainless steel, nylon or other high tensile strength biocompatible material(s). The cable is preferably about 0.2 to 2.0 millimeters in diameter. The cable loops are preferably about 20 to 35 centimeters long. A handle component 106, 108, which is preferably made of plastic, is seen at the proximal end of each cable loop.

Each cable loop passes through a small tube 110, 112 attached to the side of the instrument near the proximal end of the instrument and passes under a spring component 114 near the distal end of the instrument. A lumen 120 about, 4 to 15 millimeters in diameter, courses through the shaft of the tube-like instrument 122 in a longitudinal direction. The tube, which forms the shaft of the instrument is preferably made of stainless steel, plastic, polyethylene, or other biocompatible material(s). The tube is preferably about 0.4 to 4 millimeters thick. The shaft of the instrument could be made of multiple tubes, which could be made of different materials, placed within each other. For example, the inner tube 120 could be made of stainless steel with a thickness on the order of 0.5 millimeters.

The outer tube 122 could be made of plastic and be about 1 to 3 millimeters thick. The inner and outer tubes may be preferably fastened together with a biocompatible adhesive or weld. Alternatively, the tubes could be press fit together or not fastened to each other. A spring 114 is preferably placed over the inner tube 120 but not the outer tube 122. The spring 114 is preferably made of metal, such as stainless steel and is about 0.5 to 3 centimeters long and made of material that is about 0.4 to 2 millimeters thick. The distal ends of the cable loops pass under the spring and extend away from the sides of the instrument. The proximal end 109 of the instrument is configured to releasably attach to other instruments, such as suture cutter and suture welder instruments. As a specific example, the invention may fit over the shaft of a suture welding instrument (Tornier, Edina, Minn.).

FIG. 1B is a lateral view of the embodiment of the invention drawn in FIG. 1A. An elongate slot-like opening 118 is seen along the side of the distal end of the instrument. The proximal and middle portions of the slot are preferably 0.1 to 0.5 millimeters wide and 1 to 6 millimeters long. The projections or legs 115, 117 on either side of the slot taper distally to increase the width of the distal end of the slot.

FIG. 1C is an exploded anterior view of the embodiment of the invention drawn in FIG. 1A. The slotted distal component 119 is threaded onto the inner tube 120 of the shaft of device. Alternative fastening methods could be used to attach the two components. The spring is passed over the distal ends of the cable loops and the inner tube. A washer 124 is seen between the distal end of the spring and the slotted component.

FIG. 1D is an anterior view of the embodiment of the invention drawn in FIG. 1C, including a flexible longitudinal fixation component 130, and tissue. The flexible longitudinal fixation component 130, such as a suture, was passed through tissue on either side of an opening in the tissue. The flexible longitudinal fixation component is preferably passed through tissue with a needle, which was removed after passing the flexible longitudinal fixation component through tissue. The flexible longitudinal fixation component is preferably made of weldable suture, such as nylon or AxyaFlex (Tornier, Edina, Minn.). The suture is preferably USP size 6-0 to #5.

The first end of the suture was aligned with the slots in the distal end of the instrument then pulled into the slots. The press-fit between the suture and the sides of the slots holds the suture in the slot. The procedure is preferably done with laparoscopic instruments or a robotic system such as the da Vinci system. The first end of the suture was then passed through the opening in the cable loop on the left side of the drawing. The second end of the suture was aligned with the slots, in the opposite direction of the first end of the suture, then pulled into the slot and passed through the second cable loop.

FIG. 1E is an anterior view of the embodiment of the invention drawn in FIG. 1F and portions of a suture welding tool 140. The proximal ends of the cable loops were pulled in a proximal direction, which pulled the ends of the suture through the longitudinal opening of the spring and between such opening of the spring and the inner tube of the device. The sutures also pass between the distal end of the spring and the washer and between the proximal end of the spring and the outer tube.

The shaft of the welding tool was passed through the lumen of the instrument. The jaws 142, 144 of the welding tool, which are seen extending through the opening in the distal end of the novel instrument, were clamped over the portions of the suture previously held in the slots of the novel instrument. The invention aligns the sutures and holds the sutures in the position necessary for proper placement in the jaws of the welding tool. The welding tool was pushed in a distal direction after clamping the sutures, which pulled the sutures from the slots in the tool. The sutures can be pulled through the provisionally clamped jaws of the welding tool.

FIG. 1F is an anterior view of the embodiment of the invention drawn in FIG. 1E. The ends of the suture were pulled in a proximal direction, preferably with laparoscopic or robotic/da Vinci instruments, which pulls the suture under the spring, through the jaws of the welding tool, through the tissue, which pulls the tissue together. Friction between the proximal end of the spring and the outer tube, between the distal end of the spring and the washer, and possibly between the inside of the spring and the outside of the inner tube maintain tension on the suture. Surgeons can repeatedly pull on the ends of the suture until the desired tension and tissue closure are achieved. The suture holding feature also immobilizes the suture during the welding process, which assures good fusion between the welded portions of the suture. The cutting tool is seen rotated about the shaft of the welding tool relative to FIG. 1E. Further rotation of the tube-like cutting tool over the shaft of the welding tool cuts the ends of the suture proximal to the weld.

The free portions of the sutures, after cutting the suture, are held between the spring and the inner tube of the instrument, which facilitates removing those portions of the suture from patients. The instrument is removed from the patient, preferably by pulling it out of a laparoscopic portal, cannula, or trocar then prepared for repeat use by passing the cable loops under the spring. The distal component can be temporarily removed from the instrument to facilitate such placement of the cable loops. The suture tension holding capacity of the instrument can preferably adjusted by changing the stiffness of the spring, the length of the spring, or by changing the distance the distal component is threaded onto the inner tube of the instrument. For example, the device can be configured to hold higher tension on the sutures by using a stiffer or longer spring, or by threading the distal component further along the inner tube, which compresses the spring. The instrument, by aligning and holding the sutures for placement in the jaws of a welding tool, allowing adjustment of suture tension, maintaining suture tension, immobilizing the sutures during the welding process, and removing the free portions of the cut sutures, facilitates laparoscopic or robotic/da Vinci assisted suture welding, which enables knotless suture fixation. FIG. 1G is a lateral view of the embodiment of the invention drawn in FIG. 1F.

FIG. 2A is an anterior view of an alternative embodiment of the invention drawn in FIG. 1A. The instrument is similar in size and shape to the invention described in FIG. 1A, and both instruments may be made of similar materials. The distal end of the suture was pulled under the spring with a cable loop, which was discarded after such placement of the distal end of the suture. The distal portion of the suture was then press fit into the slots in the distal end of the instrument. The loaded instrument is preferably passed through laparoscopic cannulas for laparoscopic or robotic/da Vinci assisted surgery. The invention performs the first steps of the method taught in FIGS. 1A-1G before the loaded instrument is placed in patients, which reduces the number of steps surgeons must perform to weld sutures in patients.

FIG. 2B is an anterior view of the distal end of the embodiment of the invention drawn in FIG. 2A and tissue. The needle and second portion of the suture were passed through tissue then the suture was press fit into the slots, in the opposite direction of the first portion of the suture. The needle was passed through the opening in the cable loop and the suture was cut, which released the needle. The suture is then tightened and welded using the method taught in FIGS. 1A-1G. The instrument can be reloaded with suture for additional suture welds in the same patient.

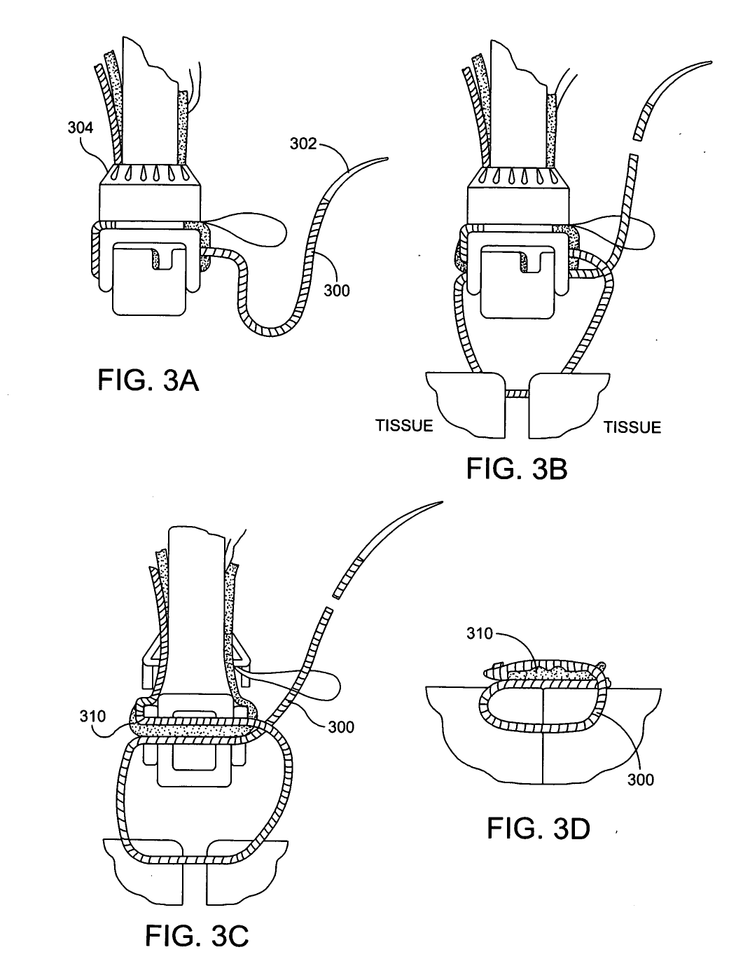

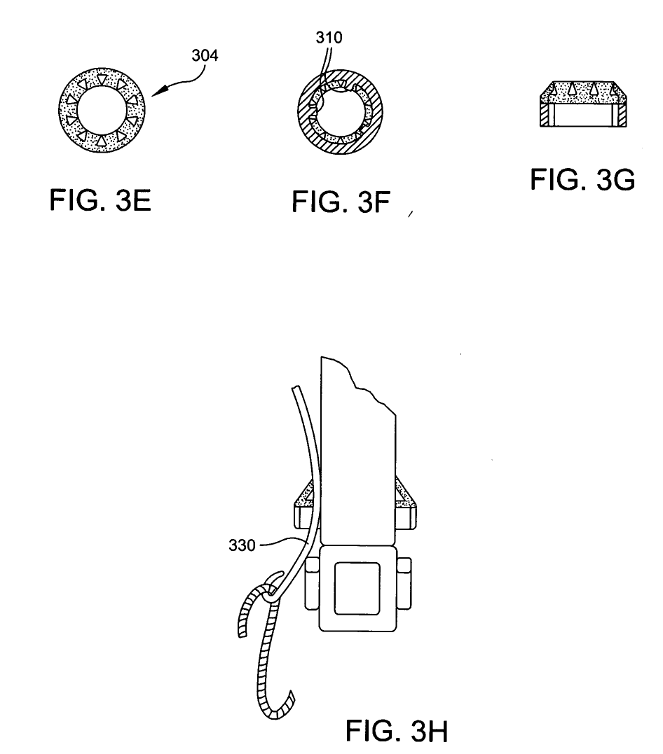

FIG. 3A is a lateral view of the distal end of an alternative embodiment of the invention drawn in FIG. 2A. The proximal end of a first flexible longitudinal fixation component, such as a suture, was pulled under a flexible ring component near the distal end of the instrument with a cable loop that was discarded. The suture was then pulled into slots in the distal end of the instrument and into open jaws of a welding tool, which was passed through the lumen of the shaft of the novel instrument. A needle 302 is seen on the distal end of the suture 300. The proximal and distal ends of a second suture were pulled under the flexible component with cable loops that were discarded then the central portion of that suture was pulled into the slots in the instrument and into the jaws of the welding tool. A portion of a cable loop is seen passing under the flexible ring.

The instrument is preferably similar in size to the invention drawn in FIG. 1A and both devices may be made of similar materials. The flexible ring is preferably elastic and made of stainless steel, titanium, plastic, or other biocompatible materials. The diameters of the sutures are wider than the space between the inner side of the ring and the outer surface of the shaft of the instrument and the diameter of the cable is preferably smaller than the width of such space. The invention holds the sutures in such location and maintains tension on the sutures. The shaft of the loaded instrument is preferably passed through trocars for laparoscopic or robotic/da Vinci aided surgeries.

FIG. 3B is an anterior view of the embodiment of the invention drawn in FIG. 3A and tissue. The needle was passed through tissue then through the opening in the cable loop. The suture, just proximal to the needle, was pulled into the slots in the distal end of the instrument and into the open jaws of the welding instrument. The distal end of the suture was cut to release and remove the needle. The wire loop pulls the distal end of the suture under the flexible ring in the next step in the procedure then the surgeon pulls on the ends of the first suture and may pull on the ends of the second suture and the sutures are welded in cut using the invention described in FIGS. 1A-1G.

FIG. 3C is an anterior view of a longitudinal cross section of the embodiment of the invention drawn in FIG. 3B. The first suture 300, which is seen in the jaws of the welding tool on either side of the second suture 310, is preferably made of high melting temperature material. The second suture 310, seen in the jaws of the welding tool between portions of the first suture, is preferably made of low melting temperature material. For example, the first suture could preferably be a size 0 multifilament polyglycolic acid suture (Dexon H, Covidien, Dubin, Ireland), which melts at about 230° C. and the second suture could preferably be a size 0 Vicryl multifilament or monofilament suture (Ethicon, Somerville, N.J.), which melts at 150° C. to 200° C. The temperature of the welder is set below 230° C. and above 150° C. to 200° C., to melt the Vicryl suture but not the polyglycolic acid suture.

Pressure from the welder forces the melted Vicryl material into the spaces between the fibers, around the fibers, or over the fibers of the polyglycolic acid suture. The sutures are welded or bonded together when the melted Vicryl material cools. Such welded suture absorbs in vivo in about 60 to 70 days. Other combinations of sutures could be used in other embodiments of the invention. For example, the high temperature suture could be made of polyglycolic acid or be a Vicryl suture and the low melting temperature suture could be a TephaFLEX (melting temperature 60° C., Tepha Medical Devices, Lexington, Mass.) or a PDS II (melting temperature 106° C., Ethicon, Somerville, N.J.). The temperature of the welder is set between the high melting temperature suture and low temperature suture combination selected.

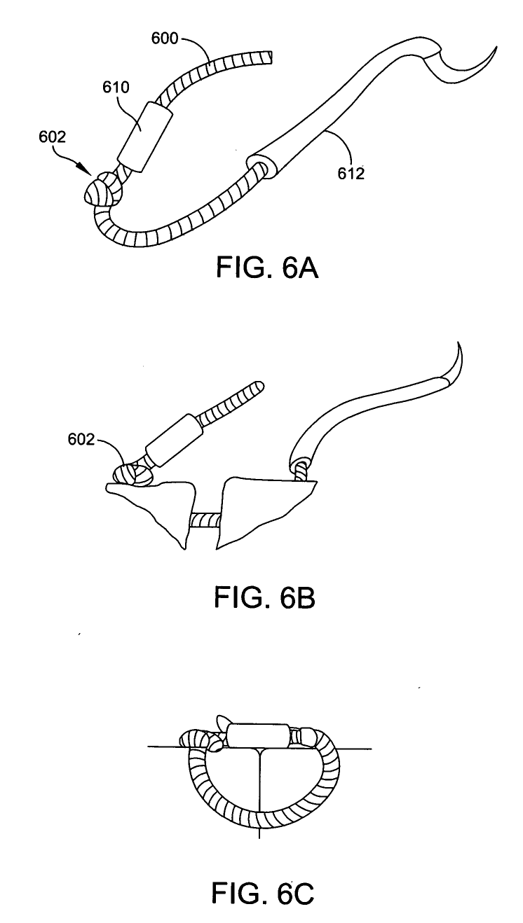

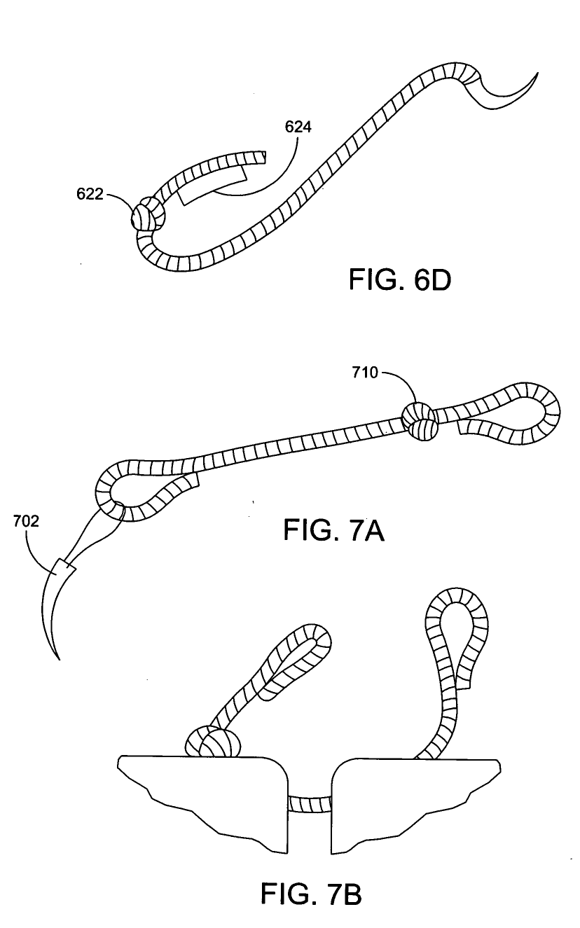

As described in the text of FIG. 6 of my co-pending application Ser. No. 61/420,837 entitled “Weldable Flexible Longitudinal Fixation Components,” the invention preferably limits materials with absorption times longer than 70 days and low melting temperatures, such as TephaFLEX and PDS II to the abdominal side of the closed vaginal cuff, while the rapid absorption and high melting temperature suture passes over the abdominal and the vaginal side of the closed vaginal cuff. Other combinations of high and low melting temperature suture materials and other suture sizes can be used in alternative embodiments of the invention. For example, the high melting temperature suture could be size 0 and the low melting temperature suture could be size 1. One or both sutures selected can be made of absorbable, non-absorbable, or combinations of absorbable and non-absorbable materials.

FIG. 3D is an anterior view of a partial sagittal cross section of the tissue closed and the embodiment of the invention drawn in FIG. 3C. The low melting temperature suture 310 is seen melted between, into, and around portions of the high melting temperature suture 300. The low melting temperature material is limited to the exterior, or one side of the closed tissue.

FIG. 3E is a superior view of the flexible ring component drawn in FIG. 3A. The spaces between the superior ends of the projections from the ring are preferably smaller than the diameters of the suture and the cable. Such spaces are preferably 0.1 mm or less. The shaft of the welding tool passes through the large opening in the center of the ring. Alternatively the suture holding ring could be fastened to the distal end of the welding tool. For example, the ring component could be pinned, glued (Cyanoacrylate or other biocompatible adhesive) or threaded onto the shaft of the welding tool.

FIG. 3F is an inferior view of the flexible ring component drawn in FIG. 3E. Projections 310 from the inside of the distal end of the ring component lie against the shaft of the instrument drawn in FIG. 3A or the shaft of the welding tool. FIG. 3G is an anterior view of a longitudinal cross section of the ring component drawn in FIG. 3F. The proximal component of such ring is preferably made of stainless steel or titanium. The distal component of the ring is preferably made of plastic.

FIG. 3H is an anterior view of a longitudinal cross section of the embodiment of the invention drawn in FIG. 3A and novel tool used to pass sutures or cable loops under the ring component. The tool 330 is preferably made of a stiff wire-like material. The distal end of the tool has a loop. The proximal end of the tool is forced between the inside of ring and the shaft of the instrument or the shaft of the welding tool. The tool is then pulled from under the ring, in a proximal direction, which pulls the end of the suture or cable under the ring.

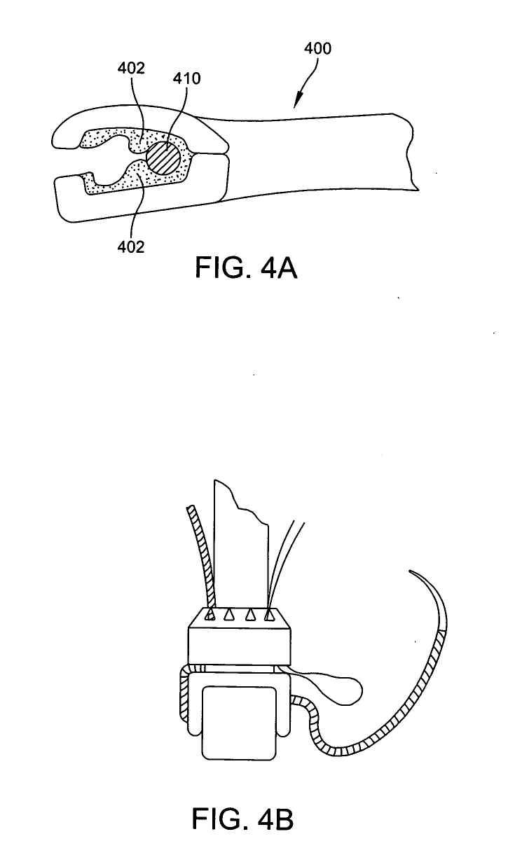

FIG. 4A is a lateral view of the embodiment of the invention drawn in FIG. 1A of my co-pending application Ser. No. 61/420,837 entitled “Weldable Flexible Longitudinal Fixation Components.” A component 402 made of low melting temperature is seen in the jaws of a welding device 400. The cross section of a first portion of a high melting temperature suture 410 is seen in the low melting temperature component. The components are preferably made of the high and low melting temperature combination of materials previously listed in this and my co-pending application.

FIG. 4B is an anterior view of the distal end of the embodiments of the invention drawn in FIGS. 3A and 4A. The distal end of the loaded welder was passed through the shaft of the tube-like component. The first end of the suture was then pulled under the flexible ring with a cable loop, which was discarded. The flexible longitudinal fixation component was then pulled into the slots in the distal end of the device. The loaded instrument is then preferably passed through a laparoscopic trocar and used as taught in FIGS. 3A-3H.

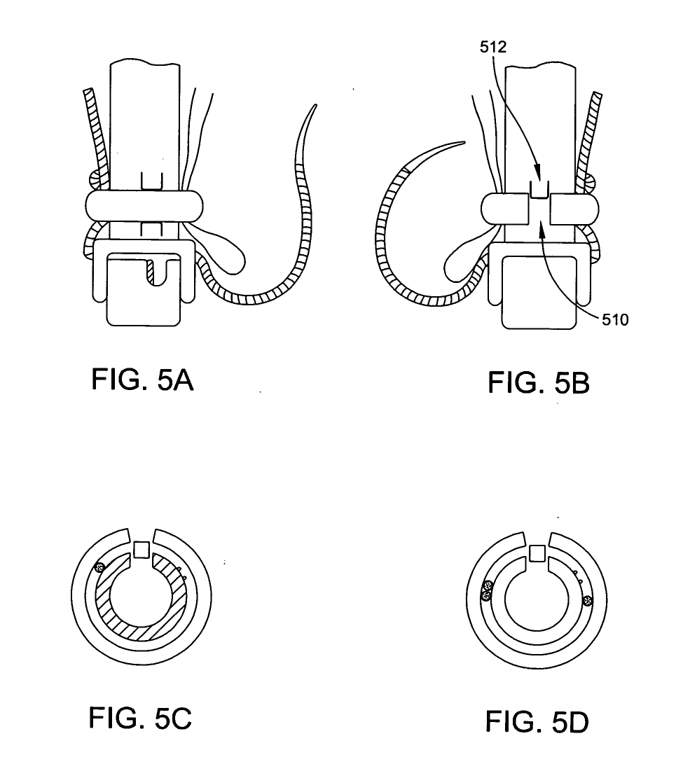

FIG. 5A is an anterior view of the distal end of an alternative embodiment of the invention drawn in FIG. 3A. The first end of a weldable flexible longitudinal fixation component was pulled beneath a flexible ring preferably with a cable loop that was discarded. A knot or other enlargement near the first end of the suture prevents the suture from passing under the ring. The suture is preferably knotted after passing it under the ring. The suture is then pulled into the slots in the distal end of the tool and into the jaws of a welding tool. A portion of a cable loop is seen passing under the flexible ring. Projections from the shaft of the tube are seen above and below the ring. The projections hold the ring near the distal end of the shaft of the tool. The instrument is similar in size to the instrument drawn in FIG. 3A and both instruments are preferably made of similar materials. The loaded instrument is preferably passed through laparoscopic trocars as taught in FIGS. 3A-3H.

FIG. 5B is a posterior view of the distal end of the embodiment of the invention drawn in FIG. 5A. An opening 510 is seen in the C-shaped flexible ring. A projection 512 from the shaft of the tool is seen in the opening of the C-shaped ring. The projection prevents excessive axial rotation of the ring as cable loops and sutures are passed under the C-shaped ring.

FIG. 5C is an inferior view of a transverse cross section of the embodiment of the invention drawn in FIG. 5B. The cross section is seen just below the C-shaped ring. The suture and cable loop are seen in cross section between the ring and shaft of the instrument on the left and right sides of the drawing, respectively. The cable loops preferable pass easily through the space between the projection from the shaft of the tool and the sides of the opening in the C-shaped ring. The cable loops also preferably pass easily into the space between the ring and the shaft of the tool.

The inner diameter of the C-shape ring is preferably more than the outer diameter of the shaft of the instrument plus twice the diameter of the cable loop and smaller than outer diameter of the shaft of the instrument plus twice the diameter of the suture. For example, the space could be between 0.25 millimeters and 0.4 millimeters wide around the circumference of the shaft of the instrument. Alternatively such space could be between 0.1 millimeters and 2.0 millimeters. Friction on the suture between the inside of the ring and the outside of the shaft of the tool resist the suture sliding under the ring. Decreasing the size of such space, increasing the diameter of the suture, or increasing the stiffness of the C-shaped ring increases such friction. As previously taught, the invention maintains tension on sutures after surgeons pull on the ends of the suture to tighten the suture and close tissue.

FIG. 5D is an inferior view of a transverse cross section of alternative embodiments of the inventions drawn in 3A and 5C. Two cross sections of a low melting temperature suture, one cross section of a high melting temperature sutures, and a cable loop are seen between the C-shaped ring and the shaft of the tool. The invention holds the sutures for welding as taught in the text of FIGS. 3A-3H and 5A-5C.

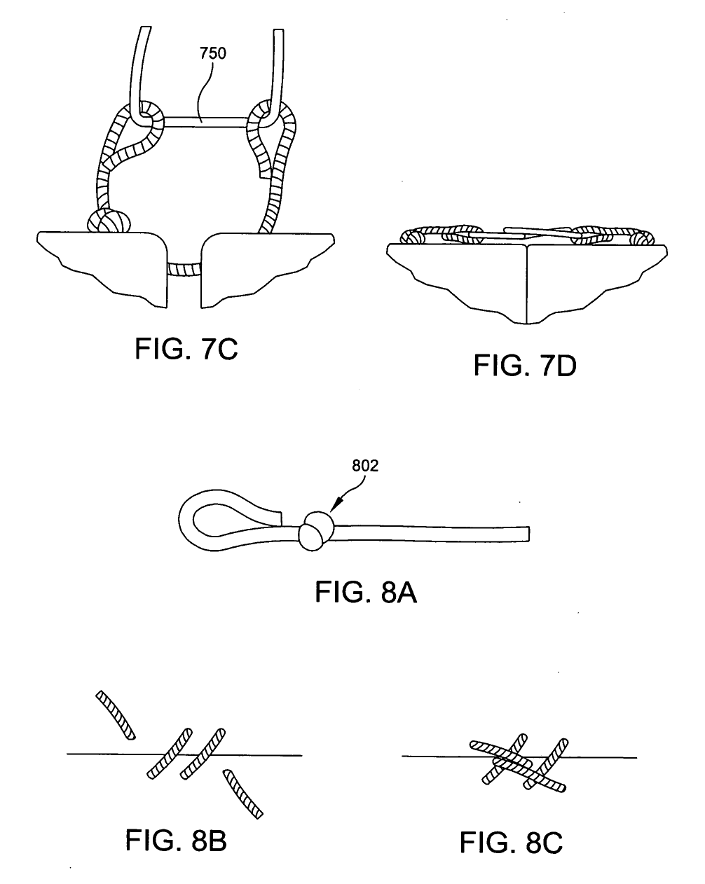

FIG. 6A is lateral view of an alternative embodiment of the inventions drawn in FIGS. 7A-7E of my co-pending application Ser. No. 61/420,837 entitled “Weldable Flexible Longitudinal Fixation Components” and FIG. 5A of this application. A knot or other enlargement 602 in a high melting temperature flexible longitudinal fixation component 600 is seen between to areas 610, 612 of low melting material, which were applied to the high melting temperature component. For example, the low melting temperature components could be made of TephaFLEX or Polydioxanone and the flexible longitudinal fixation component could be made of PGA or Polyglactin 910 (Vicryl). The space between the two sheath components is preferably 1 to 4 centimeters long and most preferably about 2.5 centimeters long. Such space could be 0.5, 0.6, 0.7, 0.8, 0.9, 4.1, 4.2, 4.3, 4.4, 4.5, less than 0.5, or greater than 4.5 centimeters in alternative embodiments of the invention. FIG. 6B is a lateral view of the embodiment of the invention drawn in FIG. 6A and tissue. The large diameter of the knot 602 prevents it from easily passing through tissue, which optimizes the positions the low melting temperature components.

FIG. 6C is a lateral view of the embodiment of the invention and tissue drawn in FIG. 6B. The low melting temperature components were melted together in an optimal position outside tissue. As previously described the invention limits slow absorption or non-absorbing materials, which preferably have low melting temperatures to preferred areas exterior to the closed tissue.

FIG. 6D is a lateral view of an alternative embodiment of the invention drawn in FIG. 6A. The low melting temperature is limited to one area of a high melting temperature suture. A positioning knot 622 is seen near the low melting temperature material 624. The low melting temperature is melted into and over a second area of the high melting temperature suture after placing the suture through tissue as shown in FIG. 6B. The invention increases the area to which the suture may be welded compared to the more limited area the suture drawn in FIG. 6A can be welded.

FIG. 7A is a lateral view of an alternative embodiment of the invention drawn in FIG. 6A. The ends of a flexible longitudinal fixation component were looped and fastened, preferably by suture welding. A releasable needle 702 is seen in the distal suture loop. A knot or other enlargement 710 is seen near the proximal suture loop.

FIG. 7B is a lateral view of the embodiment of the invention drawn in FIG. 7A and tissue. The distal end of the suture was passed through tissue then the flexible longitudinal fixation component that the attached to the needle was cut to release the needle. The knot near the proximal loop in the suture prevents pulling the proximal suture loop through tissue, which could peel the weld apart. FIG. 7C is a lateral view of the embodiment of the invention drawn in FIG. 7B and tissue. The ends of a second weldable flexible longitudinal fixation component 750 were passed through the suture loops.

FIG. 7D is a lateral view of the embodiment of the invention and tissue drawn in FIG. 7C. Tension was applied to the ends of the second suture, which pulled the suture loops and tissue together then the second suture was welded. The invention enables surgeons to pass through tissue a suture of a first material, which may be pre-welded with a first device, such as an ultrasonic welder and connection of the ends or portions of that suture with a second suture that may be welded in vivo with a different device such as a thermal welder.

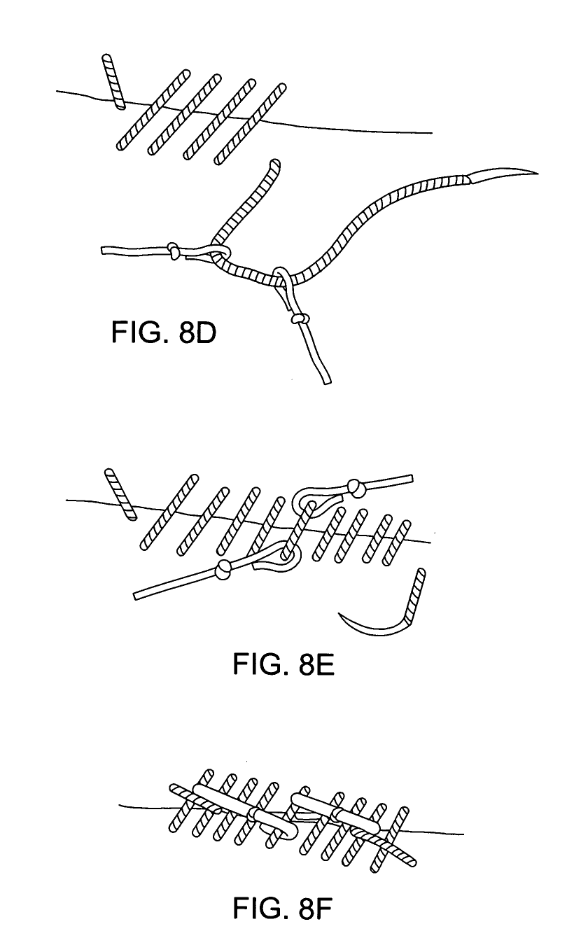

FIG. 8A is a lateral view of an alternative embodiment of the invention drawn in FIG. 7A. A loop and a knot or enlarged area 802 at seen at one end of a weldable suture. FIG. 8B is a superior view of a surgical incision through tissue and a suture used to close the incision. FIG. 8C is a superior view of the tissue and suture drawn in FIG. 8B. The ends of the “running” suture were welded together.

FIG. 8D is a superior view of a surgical incision through tissue, a suture, and the embodiment of the invention drawn in FIG. 8A. The distal end of the “running” suture was passed through the loops of two FIG. 8A devices. FIG. 8E is a superior view of the tissue, suture, and embodiment of the invention drawn in FIG. 8D. The suture was advanced through additional tissue thus capturing the looped ends of the FIG. 8A devices. FIG. 8F is a superior view of the tissue, suture, and embodiment of the invention drawn in FIG. 8E. The ends of the suture were welded to the non-loop ends of the FIG. 8A device. The invention enables surgeons to weld the ends of “running” sutures.

I claim:

1. Apparatus for welding a suture or other flexible longitudinal fixation component during a laparoscopic or robotic procedure, comprising:
an instrument having a distal end with a fixture for holding overlapping suture material; and
a welding unit with jaws that weld the overlapping suture material held by the fixture.

2. The apparatus of claim 1, wherein the instrument has a proximal end adapted for attachment to a robotically operated surgical arm.

3. The apparatus of claim 1, wherein the fixture for holding overlapping suture material includes a pair of spaced-apart projections with opposing slots.

4. The apparatus of claim 1, wherein the fixture for holding overlapping suture material includes a central bore into which the welding unit is advanced.

5. The apparatus of claim 1, wherein the fixture further includes apparatus for applying tension to the overlapping suture material held by the fixture.

6. The apparatus of claim 1, wherein the fixture further includes apparatus for applying tension to the overlapping suture material held by the fixture.

7. The apparatus of claim 1, wherein the fixture further includes two wire or cable loops through which the overlapping suture material passes, each wire or cable loop being coupled to structures that may be pulled to apply tension to the overlapping suture material held by the fixture.

8. A method of welding a suture or other flexible longitudinal fixation component during a laparoscopic or robotic procedure, comprising the steps of:
threading suture material through tissue to be closed or repaired such that two spaced-apart portions of the material are available;
providing an fixture having a distal end with a fixture for holding the two portions of the suture material in overlapping alignment; and
welding the overlapping portions of the suture material.

9. The method of claim 8, including the step of coupling the fixture to robotically operated surgical arm.

10. The method of claim 8, including the step of holding and welding the overlapping suture between a pair of spaced-apart opposing slots.

11. The method of claim 8, wherein the welding step is performed by a separate instrument received by the fixture.

12. The method of claim 8, including the step of applying tension to the overlapping suture material prior to the step of welding.

13. The method of claim 8, wherein the welding step includes melting suture material with low melting temperatures into and around portions of a rapid absorption multifilament suture.

14. The method of claim 8, wherein the welding step includes:
melting sutures with low melting temperatures into and around portions of a rapid absorption multifilament suture; and
the low-temperature suture material has an absorption time longer than that of the multifilament suture.
